(19) United States
(12) Patent Application Publication
(10) Pub. No.: US 2014/0296970 A1
(43) Pub. Date: Oct. 2, 2014

(54) Positioning Tool for Transcatheter Valve Delivery

(71) Applicant: Tendyne Holdings, Inc., Roseville, MN (US)

(72) Inventors: Craig A. Ekvall, Bethel, MN (US); Robert M. Vidlund, Forest Lake, MN (US)

(73) Assignee: Tendyne Holdings, Inc., Roseville, MN (US)

(21) Appl. No.: 14/219,591

(22) Filed: Mar. 19, 2014

Related U.S. Application Data
(60) Provisional application No. 61/807,691, filed on Apr. 2, 2013.

(51) Int. Cl. A61F 2/24

(52) U.S. Cl. CPC A61F 2/2436
USPC 623/2.11

(57) ABSTRACT

This invention relates to a positioning device for delivery of a transcatheter prosthetic heart valve that comprises a ratchet rod with reference scale for accurate positioning a valve during deployment, and methods of use thereof.

### CROSS REFERENCE TO RELATED APPLICATIONS

Not applicable.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

Not applicable.

### SEQUENCE LISTING INCLUDED AND INCORPORATED BY REFERENCE HEREIN

Not applicable.

### BACKGROUND

1. Field of the Invention

This invention relates to a positioning device for delivery of a transcatheter prosthetic heart valve that comprises a ratchet rod with reference scale for accurate positioning a valve during deployment.

2. Background of the Invention

Valvular heart disease and specifically aortic and mitral valve disease is a significant health issue in the US. Annually approximately 90,000 valve replacements are conducted in the US. Traditional valve replacement surgery, the orthotopic replacement of a heart valve, is an “open heart” surgical procedure. Briefly, the procedure necessitates surgical opening of the thorax, the initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated to the procedure largely due to the necessity for extra-corporeal circulation. Further, open heart surgery is often poorly tolerated in elderly patients.

Thus if the extra-corporeal component of the procedure could be eliminated, morbidities and cost of valve replacement therapies would he significantly reduced.

While replacement of the aortic valve in a transcatheter manner is the subject of intense investigation, lesser attention has been focused on the mitral valve. This is in part reflective of the greater level of complexity associated to the native mitral valve apparatus and thus a greater level of difficulty with regards to inserting and anchoring the replacement prosthesis.

Several designs for catheter-deployed (transcatheter) aortic valve replacement are under various stages of development. The Edwards SAPIEN® transcatheter heart valve is currently undergoing clinical trial in patients with calcific aortic valve disease who are considered high-risk for conventional open-heart valve surgery. This valve is deployable via a retrograde transarterial (transfemoral) approach or an antegrade transapical (transventricular) approach. A key aspect of the Edwards SAPIEN® and other transcatheter aortic valve replacement designs is their dependence on lateral fixation (e.g. tines) that engages the valve tissues as the primary anchoring mechanism. Such a design basically relies on circumferential friction around the valve housing or stent to prevent dislodgement during the cardiac cycle. This anchoring mechanism is facilitated by, and may somewhat depend on, a calcified aortic valve annulus. This design also requires that the valve housing or stem have a certain degree of rigidity.

At least one transcatheter mitral valve design is currently in development. The Endovalve uses a folding tripod-like design that delivers a tri-leaflet bioprosthetic valve. It is designed to be deployed from a minimally invasive transatrial approach, and could eventually be adapted to a transvenous atrial septotomy delivery. This design uses “proprietary gripping features” designed to engage the valve annulus and leaflets tissues. Thus the anchoring mechanism of this device is essentially equivalent to that used by transcatheter aortic valve replacement designs.

Various problems continue to exist in this field, including problems with insufficient articulation and sealing of the valve within the native annulus, pulmonary edema due to poor atrial drainage, perivalvular leaking around the install prosthetic valve, lack of a good fit for the prosthetic valve within the native mitral annulus, atrial tissue erosion, excess wear on the nitinol structures, interference with the aorta at the posterior side of the mitral annulus, and lack of customization, to name a few. Accordingly, there is still a need for art improved prosthetic mitral valve having a commissural sealing structure, along with a means of positioning such prosthetic valve.

### BRIEF SUMMARY OF THE INVENTION

The present invention relates to a positioning device for delivery of a transcatheter prosthetic heart valve that comprises a ratchet rod with reference scale for accurate positioning a valve during deployment.

In a preferred embodiment, there is provided a positioning device for deploying a transcatheter prosthetic cardiovascular valve in a patient, which comprises a ratchet rod having a built-in collet at a distal end for attachment to the valve, a transparent sheath having a reference scale, the ratchet rod slidably disposed within the transparent sheath and said ratchet rod having one or more markings operatively associated with reference scale, a tensioning collar attached to the transparent sheath and positioned around the slidable ratchet rod, a tensioning-release level on the tensioning collar, and a removable epicardial attachment pad attached to a proximal end of the ratchet rod.

In another embodiment, there is provided a feature wherein the transparent sheath reference scale and ratchet rod markings provide a step resolution of between about 0.5 mm and about 2.0 mm.

In another embodiment, there is provided a feature wherein the ratchet rod and pawl mechanism provide an audible feedback to a user.

In another embodiment, there is provided, a feature wherein the device has one or more radio-opaque markers thereon to facilitate positioning.

In another embodiment, there is provided a feature wherein the device fits within a surgical catheter sheath having a diameter of between about 10 Fr (3.3 mm) to about 42 Fr (14 mm).

In another embodiment, there is provided a method of tensioning a deployed transcatheter prosthetic cardiovascular valve in a patient, which comprises the step of pulling the ratchet rod to tighten a tether that extends from the valve that is surgically deployed into the mitral annulus of the patient and extends through an apical epicardial attachment point.

In another embodiment, there is provided a feature wherein the prosthetic heart valve is deployed by directly accessing the heart through an intercostal space, using an apical approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through a thoracotomy, sternotomy, or minimally-invasive thoracic, thorascopic, or transdiaphragmatic approach to enter the left (or right) ventricle, and deploying the prosthetic heart valve into the valvular annulus using the catheter delivery system.

In another embodiment, there is provided a feature wherein the prosthetic heart valve is deployed by directly accessing the heart through the intercostal space, using a lateral approach to enter the left or right ventricle, and deploying the prosthetic heart valve into the valvular annulus using the catheter delivery system.

### BRIEF DESCRIPTION OF THE DRAWINGS

FIG. 1 is a side view of a positioning tool according to the present inventive subject matter.

FIG. 2 is a view showing the ratchet rod separated from the sheath to illustrate the reference scale markings.

FIG. 3 is a side view showing the tool within a catheter and used for expelling a compressed transcatheter valve.

FIG. 4 is a side view showing the tool attached to a valve during the expelling process.

FIG. 5 is a side view of the positioning tool attached to the valve tether and where the tool is used for pulling the valve into position and the reference scale used to establish the correct tension on the tether between the valve and the apical attachment.

### DETAILED DESCRIPTION OF THE INVENTION

### Functions of the Positioning Tool

When a transcatheter valve is delivered, the compressed valve is expelled from the delivery catheter and the valve expands to its functional structure. In the case of a prosthetic mitral valve that uses an atrial cuff in combination with a ventricular tether to seat itself within the mitral annulus, when the valve is deployed into the left atrium, the valve then needs to be pulled toward the left ventricular apex to he seated within the mitral annulus, and it is then tethered to a suitable ventricular location (e.g., ventricular apex). The positioning tool is used to pull the valve down into the mitral annulus and to impart tension into the ventricular tether. The amount of tensioning force can range from that of a positioning tether (low) to that of a tensioning tether (high).

### Tethers

The tethers that are attached to the prosthetic heart valve may extend to one or more tissue anchor locations within the heart. In one preferred embodiment, the tethers extend downward through the left ventricle, exiting the left ventricle at the apex of the heart to he fastened on the epicardial surface outside of the heart. Similar anchoring is contemplated herein as it regards the tricuspid, or other valve structure requiring a prosthetic. There may be from 1 to 8 tethers which are preferably attached to the body of the valve. The positioning tool may be used for adjustment of each tether.

In another preferred embodiment, the tethers may optionally be attached to the atrial cuff to provide additional control over position, adjustment, and compliance. In this preferred embodiment, one or more tethers are optionally attached to the flared end or cuff, in addition to, or optionally, in place of, the tethers attached to the stent. By attaching to the flared end or cuff and/or the stent, an even higher degree of control over positioning, adjustment, and compliance is provided to the operator during deployment. The positioning tool may be used for adjustment of each atrial or positioning tether.

In another preferred embodiment, the tethers are optionally anchored to other tissue locations depending on the particular application of the prosthetic heart valve. In the case of a mitral valve, or the tricuspid valve, there are optionally one or more tethers anchored to one or both papillary muscles, the septum, and/or the ventricular wall.

Upon being drawn to and through the apex of the heart, the tethers may be fastened by a suitable mechanism such as tying of to a pledget or similar adjustable button-type anchoring device to inhibit retraction of the tether back into the ventricle. In a preferred embodiment, an epicardial pledget or attachment pad may be integrated directly into the toll, for instance on the ratchet rod so that once proper tension is achieved, the pad may be slid into place and surgically secured.

Further, it is contemplated that the prosthetic heart valve may optionally he deployed with a combination of installation tethers and permanent tethers, attached to either the stent or flared end or cuff, or both, the installation tethers being removed after the valve is successfully deployed. It is also contemplated that combinations of inelastic and elastic tethers may optionally be used for deployment and to provide structural and positional compliance of the valve during the cardiac cycle. The positioning tool may be used for adjustment of these tethers as well.

### Pledget/Attachment Pad

In one embodiment, to control the potential tearing of tissue at the apical entry point of the delivery system, a circular, semi-circular, or multi-part pledget is employed. The pledget may be constructed from a semi-rigid material such as PFTE felt. Prior to puncturing of the apex by the delivery system, the felt is firmly attached to the heart such that the apex is centrally located. Secondarily, the delivery system is introduced through the central area, or orifice as it may be, of the pledget. Positioned and attached in this manner, the pledget acts to control any potential tearing at the apex.

### DESCRIPTION OF FIGURES

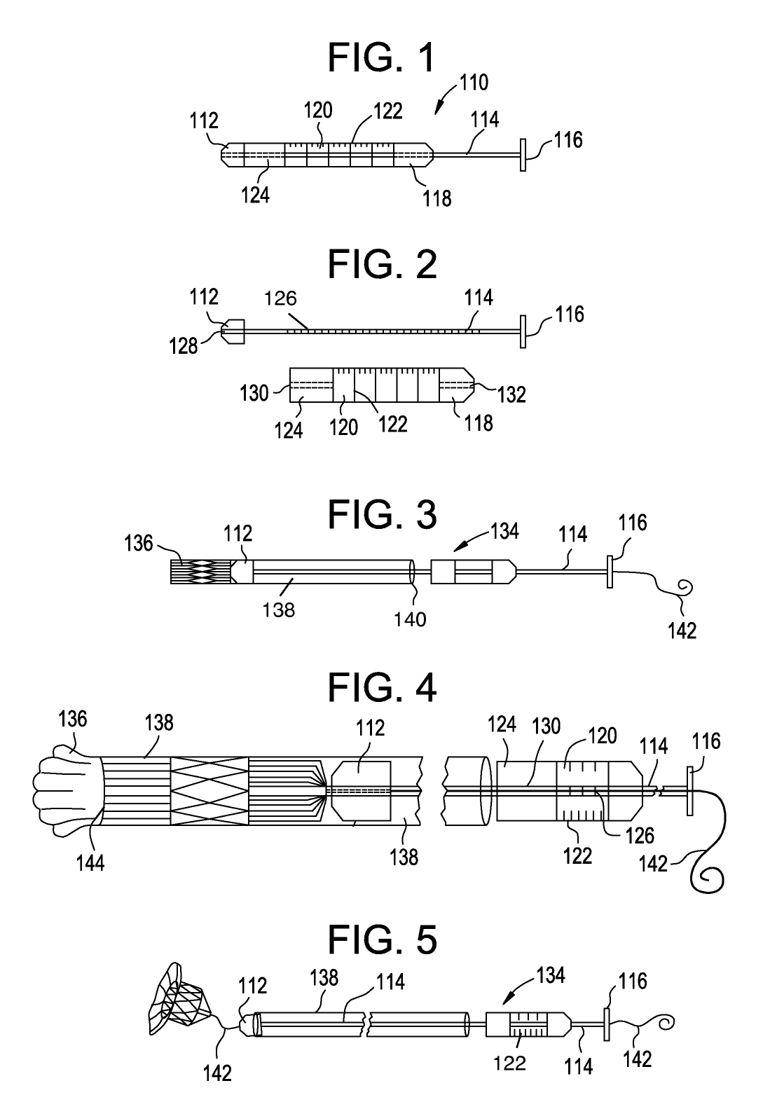

Referring now to the FIGURES, FIG. 1 is a side view of a positioning tool according to the present inventive subject matter. FIG. 1 shows tool 110 having collet 112, tensioner 124, transparent sheath 120 with reference markings 122, ratchet rod 114, and sheath support 118. FIG. 1 also shows attachment pad 116.

FIG. 2 is a view showing the ratchet rod 114 separated from the sheath 120 to illustrate the reference scale 122 and rod markings 126. FIG. 2 also shows collet 112 having aperture 128 which functions as a through-hole for an apical tether that has been attached to a valve being held by the collet 112. FIG. 2 also shows tensioner 124 having tensioner aperture 130 and sheath support 118 having sheath support aperture 132. FIG. 2 also shows attachment pad 116 attached to ratchet rod 114.

FIG. 3 is a side view showing the tool within a catheter 138 and used for expelling a compressed transcatheter valve 136. FIG. 3 shows collet 112, catheter opening 140 with tensioner/reference sheath unit 134 outside the intracardiac catheter 138. FIG. 3 also shows tension release lever 125 for releasing the tension on ratchet rod 114 for re-adjustment of tether 142.

FIG. 4 is a side view showing the tool attached to a valve during the expelling process. Valve 136 is being expelled from catheter 138 through distal catheter aperture 144 and shows how the positioning tool can used to view reference scale 122 on sheath 120 connected to tensioner 124 having tensioner aperture 130, and the movement compared to ratchet rod 114 and the rod markings 126. Collet 112, tether 142 are also shown.

FIG. 5 is a side view of the positioning tool attached to the valve tether 142 and where the tool is used for pulling the valve 136 into position using the ratchet rod 114 within sheath 138 and the reference scale 122 with rod markings 126 (not shown) being used to establish the correct tension on the tether 142 between the valve 136 and the apical attachment 116.

The references recited herein are incorporated herein in their entirety, particularly as they relate to teaching the level of ordinary skill in this art and for any disclosure necessary for the commoner understanding of the subject matter of the claimed invention, It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A positioning device for deploying a transcatheter prosthetic cardiovascular valve in a patient, which comprises a ratchet rod having a built-in collet at a distal end for attachment to the valve, a transparent sheath having a reference scale, the ratchet rod slidably disposed within the transparent sheath and said ratchet rod having one or more markings operatively associated with reference scale, a tensioning collar attached to the transparent sheath and positioned around the slidable ratchet rod, a tensioning-release lever on the tensioning collar to actuate a pawl mechanism within the tensioning collar, and a removable epicardial attachment pad attached to a proximal end of the ratchet rod.

2. The positioning device of claim 1, wherein the transparent sheath reference scale and ratchet rod markings provide a step resolution of between about 0.5 mm and about 2.0 mm.

3. The positioning device of claim 1, wherein the ratchet rod and pawl mechanism provide an audible feedback to a user.

4. The positioning device of claim 1, wherein the device has one or more radio-opaque markers thereon to facilitate positioning.

5. The positioning device of claim 1, where the device fits within a surgical catheter sheath having a diameter of between about 10 Fr (3.3 mm) to about 42 Fr (14 mm).

6. A method of tensioning a deployed transcatheter prosthetic cardiovascular valve in a patient, which comprises the step of pulling the ratchet rod to tighten a tether that extends from the valve that is surgically deployed into the mitral annulus of the patient and through an apical epicardial attachment point.
